(19) United States
(12) Patent Application Publication
(10) Pub. No.: US 2004/0101517 A1
(43) Pub. Date: May 27, 2004

(54) Blood brain barrier modulation using stressed autologous blood cells

(76) Inventors: Anthony E. Bolton, Bakewell (GB); Arkady Mandel, North York (CA)

Correspondence Address: FOLEY & HARDNER LLP, THREE PALO ALTO SQUARE, 3000 EL CAMINO REAL, SUITE 100, PALO ALTO, CA, 94306, US

(21) Appl. No.: 10/470,925

(22) PCT Filed: Feb. 1, 2002
(86) PCT No.: PCT/CA02/00127

(30) Foreign Application Priority Data
Feb. 1, 2001 (CA) 2,333,494

(51) Int. Cl. A61K 45/00; C12N 5/08

(52) U.S. Cl. 424/93.7; 435/372

(57) ABSTRACT

A method of alleviation, prophylaxis against or preconditioning to hinder the on-set and progression of a neuro-degenerative disorder, such as Alzheimer's Disease, Parkinson's Disease or senile dementia, comprises treating a patient suffering from or at risk to contract such a disorder and having impaired endothelial function at the blood vessels, with autologous stressed blood cells, to improve the performance of endothelial function at the blood brain barrier towards restoration of normal endothelial function.

### FIELD OF THE INVENTION

This invention relates to medical treatments and pharmaceutical compositions and uses. More particularly, the invention is concerned with neuro-degenerative disorders, their management and treatment.

### BACKGROUND OF THE INVENTION

Neuro-degenerative disorders such as Alzheimer's Disease, Parkinson's Disease and senile dementia, have recently come to be understood to be associated with inflammatory reactions in the brain, leading to neuronal damage. This suggests that inflammation-causing substances may be breaching the blood brain barrier, which in turn suggests that a patient suffering from a neuro-degenerative disorder may have a compromised blood-brain barrier.

The so-called “blood-brain barrier” consists essentially of the walls of the blood vessels of the brain. The brain is supplied with blood vessels (arteries, veins, capillaries, etc.), through which blood circulates to fulfill its transporting functions to the brain. The blood vessels have walls through which oxygen and other small molecules can migrate, into the brain cells and tissues. The blood vessel walls have various components including the endothelium and the smooth muscle.

The endothelium is a cellular structure which lines the blood vessels including blood vessels of the brain, and communicates with the smooth muscle layer of the blood vessel walls. Originally thought to function primarily to protect the blood vessels, the endothelium has more recently been recognized to play a more complex role, e.g. in expressing and secreting vasodilatory and vasoconstrictive components to regulate contraction and relaxation of the blood vessel and thereby play a role in regulating blood flow.

Until recently, the central nervous system (CNS) has been considered to be an immunologically privileged site to protect it from damage originating, for example, from inflammation arising in the periphery, with the blood-brain barrier restricting the entry of circulating lymphocytes. During inflammatory conditions in the CNS, immune cells immigrate into the CNS and can be detected in the CNS parenchyma and the cerebrospinal fluid. The mechanisms that regulate inflammatory cell recruitment across the blood brain barrier during CNS inflammation have not been characterized. However, endothelial dysfunction and activation may constitute a critical part of a cascade of events leading to increases in blood-brain barrier permeability to non-neural proteins, leading to inflammation and brain tissue damage. Released inflammatory cells may yield deleterious compounds or cytokines that exacerbate the inflammatory damage to metabolically compromised neurons. These inflammatory mechanisms may operate in the pathophysiology of neuro-degenerative diseases in which endothelial factors, inflammation and brain tissue damage are implicated.

The loss of well-regulated endothelial cell functioning is followed by adverse changes in a variety of physiological systems, such as the expression of adhesion molecules, maintenance of adequate blood vessel tone and overall homeostasis. In addition, endothelial dysfunction and endothelial mediated vascular inflammation may lead to breach of the blood-brain barrier, and this in turn may produce biochemical derangements that are conducive to production of β-amyloid.

β-amyloid has recently come to be understood to be one of the causes of inflammatory reactions in the brain leading to neuronal damage. Its presence in the brain is thought to indicate a compromised blood-brain barrier—either a precursor of the protein, the protein itself or cells which secrete it are crossing the blood-brain barrier in patients with neuronal damage, but not in otherwise healthy patients. Gradual accumulation of β-amyloid and perhaps other brain damaging substances from the blood mayoccurin patients with a compromised blood brain barrier, leading to inflammation, neuronal damage, and a gradual progression in the severity of the damage.

### SUMMARY OF THE INVENTION

The present invention is based on the discovery that a deficient or malfunctioning endothelium in a patient has a significant, adverse effect on the integrity or permeability (transport properties) of the blood brain barrier. Various substances, naturally present in the blood or introduced into the blood, will cross the blood brain barrier of a patient with a deficient or malfunctioning endothelium, whereas they do not cross the blood brain barrier, at least to any significant extent, when the endothelium is normal. Such substances may include neuronal inflammation-causing proteins carried by the blood, such as β-amyloid or its precursors. Over a period of time, the brain may accumulate quantities of blood borne materials such as pro-inflammatory proteins, or their metabolic products, if there is a defective endothelium at the patient's blood brain barrier. Such a gradual accumulation may underlie the gradual on-set of a neurological disorder and its gradual progression. It is generally accepted that endothelial dysfunction is rare in young patients, and that its prevalence increases with aging.

Accordingly, the present invention is a method of alleviation, prophylaxis against or preconditioning to hinder the on-set and progression of a neuro-degenerative disorder, such as Alzheimer's Disease, Parkinson's Disease, or senile dementia, which comprises treating a patient suffering from or at risk to contract such a disorder and having impaired endothelial function at the blood vessels, to improve the performance of endothelial function at the blood brain barrier towards restoration of normal endothelial function, by the administration to the patent of autologous blood cells which have been appropriately stressed in vitro. This represents a novel and innovative approach to the management and treatment of neuro-degenerative disorders.

The present invention also includes a process in which patients are diagnosed for defective endothelial function. Based upon the results of such diagnosis, a population group is selected for endothelial dysfunction contributing to a patient's neurodegenerative disorder or rendering the patient susceptible thereto. The so-selected sub-group is then treated aforesaid.

### BRIEF REFERENCE TO THE DRAWINGS

FIG. 1 and FIG. 2 of the accompanying drawings are graphical presentations of the results obtained according to Example 1 below, and

FIG. 3 is a graphical presentation of results obtained according to Example 2 below;

FIGS. 4 and 5 are graphical presentations of the results of Example 3 below.

### DESCRIPTION OF THE PREFERRED EMBODIMENTS

In accordance with preferred embodiments of the invention, a patient is treated to alter a defective endothelium towards normalization of its function by administration to the patient of autologous blood cells which have been extracorporeally stressed by subjection to appropriate amounts of oxidative stress, preferably simultaneously with exposure to ultraviolet radiation and preferably also at an elevated temperature. This process as applied to the alleviation of the symptoms of various autoimmune disorders is fully described in U.S. Pat. No. 5,980,954 Bolton, issued Nov. 9, 1999, the disclosure of which is incorporated herein by reference. Briefly, it involves extracting an aliquot of blood, e.g. 10 cc, from a patient, subjecting the aliquot extracorporeally to oxidative stress e.g. bubbling ozone/oxygen mixture through the aliquot, and simultaneously exposing the aliquot to ultraviolet radiation, at a slightly elevated temperature, e.g. 38.5° C. The stressing treatment causes stressing of the blood cells in the aliquot, to alter their cytokine profile. When these stressed cells are reinjected into the patient, they have an effect on the endothelium and tend to normalize the function of a defective endothelium. In doing so, they alter the transport properties of the blood brain barrier, bringing it back toward a normal function and therefore restoring the blood brain barrier to a condition in which it allows only those blood borne substances intended to cross the blood brain barrier, such as oxygen, nutrients, various hormones and various ions, to cross into the brain tissue and brain cells. The stressed cells, or blood components affected by the stressed cells after re-introduction of the blood aliquot into the patient, acting through the endothelium, have beneficial effects on neurological disorders, from which the patient may be suffering, at least to the extent of hindering the progression thereof, and even effecting substantial alleviation of the symptoms of the disease.

The source of the stressed blood cells for use in this invention is the patient's own blood, i.e. an aliquot of autologous blood, or a cellular fraction thereof.

The terms “aliquot”, “aliquot of blood” or similar terms used herein include whole blood, separated cellular fractions of the blood including platelets, separated non-cellular fractions of the blood including plasma, plasma components and combinations thereof. Preferably, in human patients, the volume of the aliquot is up to about 400 ml, preferably from about 0.1 to about 100 ml, more preferably from about 1 to about 15 ml, even more preferably from about 8 to about 12 ml, and most preferably about 10 ml. The effect of the stressor or the combination of stressors is to modify the blood, and/or the cellular or non-cellular fractions thereof, contained in the aliquot. The modified aliquot is then re-introduced into the subject's body by any suitable method, most preferably intramuscular injection, but also including subcutaneous injection, intraperitoneal injection, intra-arterial injection, intravenous injection and oral administration.

According to a preferred process of the present invention, an aliquot of blood is extracted from the human patient, and the aliquot of blood is treated ex vivo, simultaneously or sequentially, with the aforementioned stressors. Then it is injected back into the same subject. Preferably a combination of both of the aforementioned stressors is used.

Preferably also, the aliquot of blood is in addition subjected to mechanical stress. Such mechanical stress is suitably that applied to the aliquot of blood by extraction of the blood aliquot through a conventional blood extraction needle, or a substantially equivalent mechanical stress, applied shortly before the other chosen stressors are applied to the blood aliquot. This mechanical stress may be supplemented by the mechanical stress exerted on the blood aliquot by bubbling gases through it, such as ozone/oxygen mixtures, as described below. Optionally also, a temperature stressor may be applied to the blood aliquot, simultaneously or sequentially with the other stressors, i.e. a temperature at, above or below body temperature.

The optionally applied temperature stressor either warms the aliquot being treated to a temperature above normal body temperature or cools the aliquot below normal body temperature. The temperature is selected so that the temperature stressor does not cause excessive hemolysis in the blood contained in the aliquot and so that, when the treated aliquot is injected into a subject, the desired effect will be achieved, without development of significant adverse side effects. Preferably, the temperature stressor is applied so that the temperature of all or a part of the aliquot is up to about 55° C., and more preferably in the range of from about −5° C. to about 55° C.

In some preferred embodiments of the invention, the temperature of the aliquot is raised above normal body temperature, such that the mean temperature of the aliquot does not exceed a temperature of about 55° C., more preferably from about 40° C. to about 50° C., even more preferably from about 40° C. to about 44° C., and most preferably about 42.5±1° C.

In other preferred embodiments, the aliquot is cooled below normal body temperature such that the mean temperature of the aliquot is within the range of from about 4° C. to about 36.5° C., more preferably from about 10° C. to about 30° C., and even more preferably from about 15° C. to about 25° C.

The oxidative environment stressor can be the application to the aliquot of solid, liquid or gaseous oxidizing agents. Preferably, it involves exposing the aliquot to a mixture of medical grade oxygen and ozone gas, most preferably by applying to the aliquot medical grade oxygen gas having ozone as a component therein. The ozone content of the gas stream and the flow rate of the gas stream are preferably selected such that the amount of ozone introduced to the blood aliquot, either on its own or in combination with one of the other stressors, does not give rise to excessive levels of cell damage, and so that, when the treated aliquot is injected into a subject, the desired effect will be achieved, without development of significant adverse side effects. Suitably, the gas stream has an ozone content of up to about 300 μg/ml, preferably up to about 100 μg/ml, more preferably about 30 μg/ml, even more preferably up to about 20 μg/ml, particularly preferably from about 10 μg/ml to about 20 μg/ml, and most preferably about 14.5±1.0 μg/ml. The gas stream is suitably supplied to the aliquot at a rate of up to about 2.0 litres/min, preferably up to about 0.5 litres/min, more preferably up to about 0.4 litres/min, even more preferably up to about 0.33 litres/min, and most preferably about 0.24±0.024 litres/min. The lower limit of the flow rate of the gas stream is preferably not lower than 0.01 litres/min, more preferably not lower than 0.1 litres/min, and even more preferably not lower than 0.2 litres/min, all rates at STP.

The ultraviolet light stressor is suitably applied by irradiating the aliquot under treatment from a source of UV light. Preferred UV sources are UV lamps emitting W-C band wavelengths, i.e. at wavelengths shorter than about 280 nm. Ultraviolet light corresponding to standard UV-A (wavelengths from about 315 to about 400 nm) and UV-B (wavelengths from about 280 to about 315) sources can also be used. As in the case of the oxidative stressor, the UV dose should be selected, on its own or in combination of the other chosen stressor(s), so that excessive amounts of cell damage do not occur, and so that, when the treated aliquot is injected into a subject, the desired effect will be achieved. For example, an appropriate dosage of such UV light, can be obtained from up to eight lamps arranged to be exposed to the sample container holding the aliquot, operated at an intensity to deliver a total UV light energy at 253.7 nm at the surface of the blood of from about 0.025 to about 10 joules/cm², preferably from about 0.1 to about 3.0 joules/cm². Such a treatment, applied in combination with the oxidative environment stressor, provides a modified blood aliquot which is ready for injection into the subject.

It is preferred to subject the aliquot to the oxidative environment stressor, the UV light stressor and the temperature stressor simultaneously, following the subjection of the aliquot to the mechanical stress, e.g. by extraction of the blood from the patient. Thus, the aliquot may be maintained at a predetermined temperature above or below body temperature while the oxygen/ozone gas mixture is applied thereto and while it is irradiated with ultraviolet light.

The time for which the aliquot is subjected to the stressors is normally within the time range of from about 0.5 minutes up to about 60 minutes. The time depends to some extent upon the chosen combination of stressors. When UV light is used, the intensity of the UV light may affect the preferred time. The chosen temperature level may also affect the preferred time. When oxidative environment in the form of a gaseous mixture of oxygen and ozone applied to the aliquot is chosen as one of the two stressors, the concentration of the oxidizing agent and the rate at which it is supplied to the aliquot may affect the preferred temperature. Some experimentation, well within the ordinary skill of the art, to establish optimum times maybe necessary on the part of the operator, once the other stressor levels have been set. Under most stressor conditions, preferred times will be in the approximate range of from about 2 to about 5 minutes, more preferably about 3 minutes. The starting blood temperature, and the rate at which it can be warmed or cooled to a predetermined temperature, tends to vary from subject to subject. Warming is suitably by use of one or more infrared lamps placed adjacent to the aliquot container. Other methods of warming can also be adopted.

As noted, it is preferred to subject the aliquot of blood to a mechanical stressor, as well as the chosen stressor(s) discussed above. Extraction of the blood aliquot from the patient through an injection needle constitutes the most convenient way of obtaining the aliquot for further extracorporeal treatment, and this extraction procedure imparts a suitable mechanical stress to the blood aliquot. The mechanical stressor may be supplemented by subsequent processing, for example the additional mechanical shear stress caused by bubbling as the oxidative stressor is applied.

In the practice of the preferred process of the present invention, the blood aliquot may be treated with the heat, UV light and oxidative environment stressors using an apparatus of the type described in aforementioned U.S. Pat. No. 4,968,483 to Mueller. The aliquot is placed in a suitable, sterile container, which is fitted into the machine. A UV-permeable container is used and the UV lamps are switched on for a fixed period before the other stressor is applied, to allow the output of the UV lamps to stabilize. When a temperature stressor is used combination, the UV lamps are typically on while the temperature of the aliquot is adjusted to the predetermined value, e.g. 42.5±1° C. Four UV lamps are suitably used, placed around the container.

The above treatment to improve endothelial function and hence exert beneficial effects on neurological disorders may be used in combination with other treatments such as administration of one or more pharmaceuticals which have a beneficial effect on endothelial function. Such pharmaceuticals include angiotensin converting enzyme (ACE) inhibitors, angiotensin II receptor antagonists, statins, pentoxifylline, β-blockers, α-antagonists, thalidomide and calcium channel blocking drugs.

Accordingly, another aspect of the present invention, in a preferred embodiment, is the use of stressed autologous blood cells as described above in combination with an effective amount of an ACE inhibitor in preparation of a medicament for the treatment of, or alleviation of the symptoms of, Alzheimer's disease, Parkinson's disease or senile dementia in a mammalian patent suffering therefrom. More preferably, the ACE inhibitor for such use is selected from alacepril, benazepril, captopril, ceronapril, cilazapril, delapril, enalapril, enalaprilat, fosinopril, imidapril, lisinopril, moveltopril, perindopril, quinapril, ramipril, spirapril, temocapril and trandolapril.

It is known that ACE inhibitors, commonly prescribed to combat hypertension in patients through their vasodilation activity, act at least in part through action on the patient's endothelium (see for example see Taddei, S. et. al, Curr Hypertens Rep 2000 February;2(1): 64-70). A defective endothelium, responsible at least in part for the patients hypertension or other vascular disorder under treatment, is to a degree repaired or restored towards normal function by the action of the appropriate dose of ACE inhibitor. Known, useful ACE inhibitors for the present invention include alacepril, benazepril, captopril, ceronapril, cilazapril, delapril, enalapril, enalaprilat, fosinopril, imidapril, lisinopril, moveltopril, perindopril, quinapril, ramipril, spirapril, temocapril and trandolapril. The pharmaceutically acceptable salts of these drugs are also useful herein.

Appropriate dosages of ACE inhibitors for use in the present invention are largely in accordance with those normally administered in connection with treatment of hypertension, and are known to those skilled in the art and available from standard physicians' reference books.

Also known to have beneficial effects on a dysfunctional endothelium, and therefore potentially useful in combination with stressed autologous blood cells as described above in treating neurodegenerative diseases such as Alzheimer's disease, Parkinson's disease, multiple sclerosis and senile dementia, according to a preferred embodiment of the invention, are angiotensin II receptor antagonists such as candesartan, eprosartan, irbesartan, losartan and valsartan (see Cheetham, C., O'Driscoll, G., Stanton, K., Taylor, R. and Green, D., Cin. Sci. (Colch) Jan 1, 2001;100(1):13-17). The pharmaceutically acceptable salts of these drugs are also useful herein.

Appropriate dosages of angiotensin II receptor antagonists for use in the present invention are largely in accordance with those normally administered in connection with treatment of hypertension, and are known to those skilled in the art and available from standard physicians' reference books.

A defectively functioning endothelium of the blood vessels can also be improved towards normal function, with consequent alleviation of neurological degeneration conditions such as Alzheimer's Disease, Parkinson's Disease, multiple sclerosis and senile dementia, by administration to the patient suffering therefrom of a statin drug commonly prescribed as an antihyperlipidemic. Such statin drugs are inhibitors of the enzyme HMG CoA reductase, and are understood to act at least in part through endothelial effects (See Corsini, A., J. Cardiovasc Pharmacol Ther 2000 July;5(3):161-75; and Farmer, J. A., Curr Atheroscler Rep 2000 May;2(3):208-217).

Suitable such statin drugs for use in combination with stressed autologous blood cells in accordance with the present invention include atorvastatin, fluvastatin, lovastatin, simvastatin, pravastatin and cerivastatin. The pharmaceutically acceptable salts of these drugs are also useful herein. They can be used for purposes according to the present invention in dosage ranges generally similar to those used for the treatment of hyperlipidemia with these drugs, such doses being known to those skilled in the art and available from standard physicians' reference books. These are, in respect of atorvastatin, simvastatin, lovastatin, fluvastatin and pravastatin, from about 5 mg to about 200 mg daily, for an adult of normal body weight, preferably from about 10-80 mg. In respect of cerivastatin, an entirely synthetic compound, the most appropriate daily dosage is much lower, namely from about 0.1-0.8 mg. In the combination therapy of the invention, and afterwards, these dosages may be reduced. Oral administration of the statin drug, once per day, is most appropriate.

Another means for improving the function of a defective endothelium of the blood vessels, and hence treating or alleviating the symptoms of a neurological degenerative condition such as Alzheimer's Disease, Parkinson's Disease and senile dementia, is by administration of pentoxifylline to the patient suffering therefrom. Pentoxifylline is a known vasodilator drug, the full chemical of which is 3,7-dihydro-3,7-dimethyl-1-(5-oxohexyly)-1H-purine-2,6-dione. This also exerts its vasodilatory action, at least in part, by effects on the endothelium, tending towards a normalization of the function of a defective endothelium (see Kristova, V., Kriska, M., Babal, P., Djibril, M. N., Slamova, J. and Kurtansky, A., Physiol Res 2000;49(1):123-8; and Schratzberger, P, et.al. Immunopharmacology 1999 January:41(1):65-75), and is hence useful in combination with stressed autologous blood cells in the present invention. Appropriate daily dosages of pentoxifylline are generally in accordance with those commonly administered for use of the drug as a vasodilator, and are known to those skilled in the art and available from physicians' reference books.

Also potentially useful in the present invention are combinations of stressed autologous blood cells with calcium channel blocking drugs of the dihydropyridine type. These are known to exert beneficial effects on the endothelium (see Taddei, S, et.al, Curr Hypertens Rep 2000 February;2(1): 64-70), so that they are potentially useful in treating neurodegenerative diseases of the aforementioned type. Accordingly another preferred embodiment of the present invention is use of an effective amount of a dihydropyridine-type calcium channel blocker drug in combination with stressed autologous blood cells as described above in preparation of a medicament for the treatment of, or alleviation of the symptoms of, Alzheimer's disease, Parkinson's disease, or senile dementia in a mammalian patient suffering therefrom. Preferred such drugs are drug is amlodipine, aranidipine, bamidipine, benidipine, cilnidipine, efonidipine, elgodipine, felodipine, isradipine, lacidipine, lercanidipine, manidipine, nicardipine, nifedipine, nilvadipine, nimodipine, nisoldipine or nitrendipine.

A preferred process according to the present involves a step of determining whether a patient suffering from or at high risk of developing a neurodegenerative disorder is likely to benefit from a treatment according to the invention. To evaluate this, and consequently to select sub-populations of patients in need or in potential need of endothelial function improvement in association with neurodegenarative disorder treatment or prophylaxis, one method which can be adopted is a determination of the approximate level of endothelial function in the potential patient.

The proper functioning, or lack thereof, of the endothelium of a mammalian patient, at a particular location, can be tested by using a method which involves the iontophoretic introduction of acetylcholine through the skin, and measurement of its effects on superficial blood at the chosen location. Detection of impaired endothelial function by this testing means, atone location in a patient, is indicative of endothelial dysfunction elsewhere in the patient, including the blood vessels of the brain. Similarly, effecting improvement of endothelial function at that location, as determined by this methodology, is indicative of systemic endothelial function improvement, including blood vessel endothelium repair.

Acetylcholine introduced to a blood vessel which has intact, properly functioning endothelium stimulates the production and secretion of nitric oxid by the endothelium, to cause smooth muscle relaxation and vasodilation. This vasodilation can be quantified by measurement of blood flow in the vessel, e.g. by laser Doppler flowmetry. If however the endothelium is defective, the acetylcholine may act directly on the smooth muscle and cause them to contract, with resultant vasoconstriction. Clinical examination of endothelial function based on the effects of acetylcholine proceeds generally according to the methodology described by Chowienczyk et.al., “Impaired endothelium-dependent vasodilation of forearm resistance vessels in hypercholesterolaemia”, The Lancet, Vol.340, Dec. 12, 1992, p.1430. Briefly, acetylcholine is applied to the skin of the patient and a small electric current is applied across the skin between two adjacent electrodes, one positively charged and one negatively charged (iontophoresis). Acetylcholine passes through the skin with the current, to the superficial blood vessels. There the acetylcholine acts on the endothelial cells to cause vasodilation, or on the smooth muscle cells to cause vasoconstriction, depending on the state of the endothelium. Resultant blood flow is measured by laser Doppler flowmetry.

This method of diagnosing a patient and determining suitability of a potential patient for treatment or prophylaxis of neurodegenerative disorders is generally described for illustrative purposes in Example 1, given below.

Another method for diagnosing dysfunctional endothelium, and hence for selecting a population or sub-population of patients for whom the treatment and compositions of the present invention are suitable is based on the process described by Jimenez J. J., et. al., British Journal of Haemotolgy 2001 January: 112: 81-90 This method measures the release of microparticles <about 1.5 μm from endothelial cells, more specifically CD31+ and CD51+endothelial microparticles (EMPs), by flow cytometry using plasma labeled with fluorescein isothiocyanate-conjugated anti-CD31 and anti-CD51 antibodies. Elevation of CD31+EMP levels in a patient suspected of having a neurodegenerative disease above the level found in normal, healthy individuals (Minagar, A et. al., Neurology 2001 May 22; 56(10): 1319-1324), indicates acute injury to the patient's brain blood vessel endothelium. Elevation of CD51+EMP levels indicates chronic injury of endothelium. Either of these elevations indicates suitability of the patient for treatment of neurological disease according to the present invention.

Other methods of diagnosing dysfunctional endothelium are known in the art and may be used for determining the suitability of a potential patient for treatment or prophylaxis of neurodegenerative disorders as described herein.

### EXAMPLE 1

Four patients, human females ranging in age from 15 to 84 years, and all suffering from an endothelium deficiency-related condition (primary Raynaud's phenomenon) were subjected to a course of treatment of autologous stressed blood cells. Treatment was given by skilled, qualified personnel, in a medical hospital facility on an out-patient basis.

Each treatment administered to the patient involved removing a 10 ml aliquot of the patient's blood, into an apparatus as generally described in aforementioned U.S. Pat. No. 4,968,483, heating the sample to 42.5 degrees C. and exposing it to UV radiation at wavelength 253.7 nm. Upon reaching the required temperature (42.5 C), a gaseous mixture of medical grade oxygen with an ozone content of 12.5 micrograms per ml, at a flow rate of about 60 ml/min (STP) was bubbled through the sample for 3 minutes.

After the ex vivo treatment of the blood sample had been completed, the sample was injected into the respective patient via the gluteal muscle. Each patient underwent a course of of 10 such treatments over a period of 2-4 weeks, the individual treatments being spaced apart by about 1-3 days.

Subjectively, every patient reported a very significant alleviation of her Raynaud's symptoms, after completion of the course of treatments, indicative of an improvement in endothelial function.

For each patient, objective measurements of blood flow, before and after the course of treatments, were made by the iontophoretic technique using acetylchline as previously described. The iontophoretic applications and measurements were made on the patients' forearms The initial measurements on each patient were taken immediately before the first treatment. The subsequent mearurements were all taken one day after the completion of the course of ten treatments, and again on a follow-up basis two or three weeks later (visit 12). One of the four patients was given a second, subsequent course of five further treatments. Blood flow was measured by laser Doppler flowmetry.

For each measurement, a reservoir containing acetylcoline was mounted on the patient's arm with the acetylcholine in contact with the patient's skin. Electrodes were inserted into the reservoir so that a current of known but variable magnitude could be applied to the reservoir to exert an iontophoretic effect. The dose of acetylcholine applied to the skin is a function of the time of acetylcholine-skin contact and the voltage applied between the electrodes, thereby giving a dose in arbitrary units.

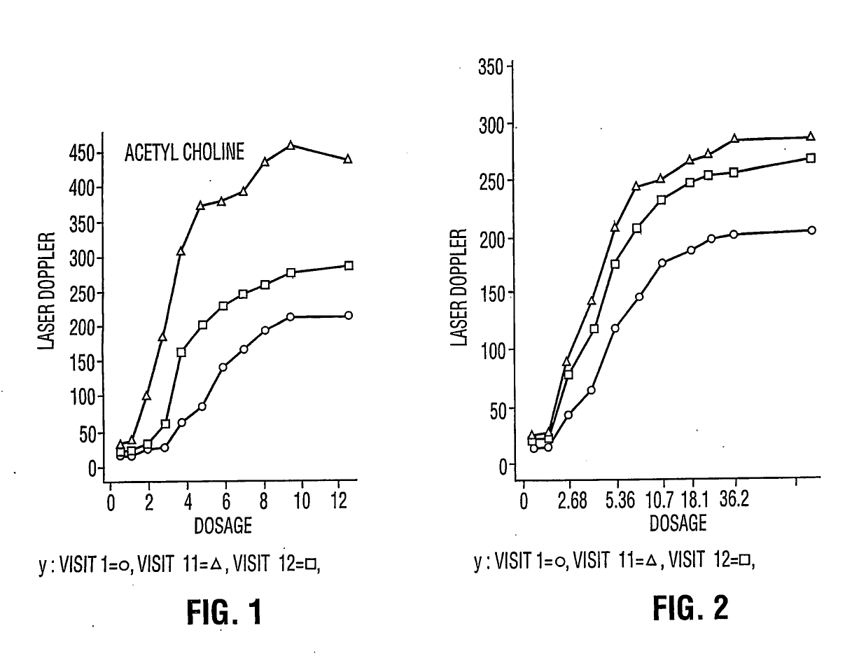

FIG. 1 presents a graph of observed laser Doppler flow of blood against dosage, in arbitrary units, determined as above, for one representative patient.

The duration of the iontophoresis was arbitrarily divided into various equal time intervals or epochs. The mean flow at each epoch is plotted against time, with the mean plotted at the mid time point of each epoch. The curve denoted by circles is that obtained from testing conducted before the first treatment, i. on the patient's first visit. As shown, blood flow increases in a generally sigmoidal fashion as the acetyl choline dosage (function of contact time and applied iontrophoretic voltage) increases. The curve denoted by triangles is that obtained in a similar manner, on the patient's 11^{th} visit, one day after the conclusion of the course of ten treatments. The curve denoted by squares is that obtained on the patient's 12^{th} visit, twenty eight days after the 11^{th} visit. Effectively, these are dose response curves. A significant increase in blood flow in response to acetyl-choline, indicative of an enhanced endothelial function, after the course of treatment, is evident from these curves.

All four of the patients treated showed essentially similar results, those presented on FIG. 1 being representative, and from a single patient. FIG. 2 of the accompanying drawings shows similar curves to FIG. 1, but derived from the means of the measured blood flows of all four of the patients. As in the case of the FIG. 1 curves from the one patient, the curves denoted by circles are the mean blood flow values, at various, increasing doses of acetylcholine from the four patients before the first treatment. The curves denoted by triangles are mean values from one day after the conclusion of the course of treatments. The curves denoted by squares are mean values from twenty eight days after the conclusion of the course of treatments. The dosage response trend is clearly apparent from the curves presented as FIG. 2.

The iontophoresis data obtained from all four patients as described above, was subjected to statistical analysis, using the data of each of the four patients obtained before any treatment, and the data obtained from all four patients two to three to four weeks after completion of the course of 10 treatments (visit 12).

As noted above, in obtaining the curves shown on FIG. 1, the mean flow at each epoch is plotted against time, with the mean plotted at the mid time point of each epoch. Since the graphs indicat that the flow increased in a sigmoid fashion, the slope of the increase was calculated, in each case, using the mean flows from the epoch with a curve starting to rise, to the point where the curve started to become asymptotic. The regression analyses used to calculate these slopes all accounted for greater than 85% of the variation, and were therefore considered a very good fit. There was also calculated a total area under the curve (AUC) from the point where the curve started to rise, to epoch 10. The maximum recorded mean flow and the area under the curve during epoch 11 were also analyzed.

Table 1 summarizes these results. It indicates that the increase in flow in response to acetylcholine was higher post treatment, since the maximum flow, the AUC during the increase and the AUC in epoch 11 were higher post treatment, to a statistically significant extent, even on the basis of four patients (the P value being 0.012, 0.020 and 0.040 respectively). The slope was also greater, but not significantly so.

TABLE 1
SUMMARY OF ANALYSIS OF IONTOPHORESIS DATA

| | | | | Visit 12 After | | | | |
|---|---|---|---|---|---|---|---|---|
| | | Baseline Before | | Course of | | Visit 12 | 95% | P Value |
| | | Treatment | | Treatment | | Minus | Confidence | From T- |
| Drug | Variable | Mean | SD | Mean | SD | Baseline | Interval | Test |
| Acetyl | Slope of | 36.55 | 17.360 | 48.1 | 12.065 | 12.26 | −1.56, 26.08 | 0.067 |
| Choline | increase | | | | | | | |
| Acetyl | Maximum | 211.4 | 40.67 | 272.3 | 22.68 | 60.93 | 25.96, 95.89 | 0.012 |
| Choline | Flow | | | | | | | |
| Acetyl | AUC during | 80695 | 24238.1 | 106708 | 15247.0 | 26013 | 7899, 44127 | 0.020 |
| Choline | increase | | | | | | | |
| Acetyl | AUC in | 64139 | 13167.0 | 83765 | 6622.3 | 19626 | 2106, 37145 | 0.040 |
| Choline | epoch 11 | | | | | | | |

### EXAMPLE 2

This experiment investigated the effect of pre-administration of stressed autologous blood cells on lipopolysaccharide (LPS) induced inhibition of long-term potentiation (LTP) in the hippocampus, in an animal model. Long-term potentiation is a form of synaptic plasticity and is thought to be the biological substrate for learning and memory.

The experimental model was inbred Wistar rats, and involves electrophysiological recording of the excitatory post-synaptic potential (EPSP) following tetanic stimulation. The synaptic activity of a specific neuronal pathway in the hippocampus, the perforant pathway, is measured. EPSP is a functional measure of post-synaptic neurotransmitter release.

The ability of the hippocampus to sustain LTP is impaired in aged rats, stressed rats and following bacterial infection. The latter can be mimicked by intraperitoneal injection of LPS, which, as well as resulting in impairment of LTP, is also associated with an increase in the levels and expression of the pro-inflammatory cytokine IL-1β in the hippocampus.

Four groups of eight animals were investigated. Half the animals were administered 0.15 ml of stressed donor rat blood intramuscularly (equivalent to autologous blood ini this inbred strain), on days-14, -13, and -1 prior to the experimental procedure. The blood was stressed as follows:

Whole blood was obtained from inbred Wistar rats, by extraction from a main artery through an injection needle, and treated with an anti-coagulant. An aliquot of this was subjected to the process described below, to obtain treated blood. The remainder was left untreated, for use in control experiments. Since these rats are genetically identical, the administration of the treated blood to others of the group is equivalent to administration of the treated blood to the donor animal.

To obtain treated blood, the selected aliquot, in a sterile, UV-transmissive container, was treated simultaneously with a gaseous oxygen/ozone mixture and ultraviolet light at elevated temperature using an apparatus as generally described in aforementioned U.S. Pat. No. 4,968,483 Mueller et.al. Specifically, 12 ml of citrated blood was transferred to a sterile, low density polyethylene vessel (more specifically, a Vasogen VC7002 Blood Container) for ex vivo treatment with stressors according to the invention. Using an apparatus as described in the aforementioned Mueller patent (more specifically, a Vasogen VC7001 apparatus), the blood was heated to 42.5±1° C. and at that temperature irradiated with UV light principally at a wavelength of 253.7 nm, while oxygen/ozone gas mixture was bubbled through the blood to provide the oxidative environment and to facilitate exposure of the blood to UV. The constitution of the gas mixture was 14.5±1.0 μg ozone/ml, with the remainder of the mixture comprising medical grade oxygen. The gas mixture was bubbled through the aliquot at a rate of 240±24 ml/min for a period of 3 minutes.

Control animals were administered untreated blood. On day 0, the animals were anaesthetized and injected with either saline or LPS (0.1 ml per kg) intraperitoneally, to give four groups:

1. Saline, untreated blood;

2. LPS, untreated blood;

3. Saline, treated blood;

4. LPS, treated blood.

Three hours later, electrodes were inserted and the electrophysiology experiment performed. The rats were then sacrificed by decapitation, the hippocampus and cortex were dissected on ice, sliced and frozen in 10% DMSO. Serum was prepared from the peripheral blood and stored frozen.

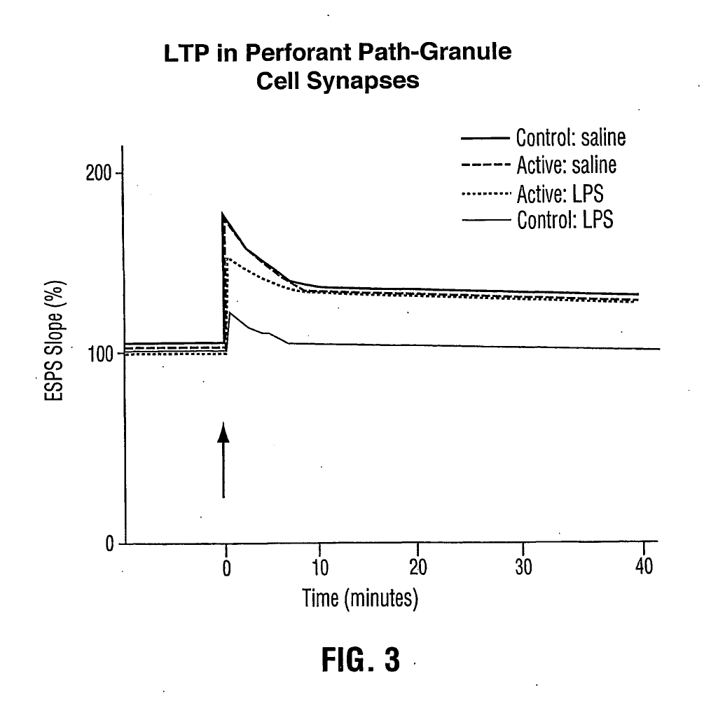

The results are shown graphically on the accompanying FIG. 3. This shows the slope of the EPSP before and after tetanic stimulation (arrow). It is to be noted that, in animals injected with saline (open squares), there is potentiation of the response (EPSP does not return to pre-tetanic baseline over a 40 minute period), whereas in animals injected with LPS (open triangles) there is no potentiation of the response. In stressed cell treated animals given LPS (closed triangles), the LTP is restored to control levels and in saline-injected animals given stresed-cell therapy thereis no difference compared to saline-control animals.

The results of this experiment show that pretreatment of animals with a course of three injections of the treated blood containing stressed cells protects the hippocampus against the loss of LTP resulting from LPS administration.

The mechanism of this protection relates at least in part to the reduced formation of LPS induced inflammation in the brains of the rats in the experiment, a mechanism that is supported by the data from the use of stressed cell administration to a patient for pre-conditioning against ischemia/reperfusion injury (see U.S. Pat. No. 6,136,308). The stressed cell therapy lowered LPS induced inflammation in the brain and gave improvement in blood brain barrier function even in normal animals, and thus this beneficial effect has the ability to cross the blood brain barrier. Lowered LPS induced inflammation and improvement in the blood brain barrier function present an attractive explanation of the observed beneficial effects of the stressed cell therapy on the endothelium.

### EXAMPLE 3

Improvement in endothelial function in the arterial system of a mammal, namely Watanabe rabbits, by use of the present invention was demonstrated.

Two groups of female Waatanabe rabbits, 7-8 months old, were selected, 10 animals in each group. Group A was given a course of treatment in which 10 ml of blood was drawn from the ear vein, treated (stressed) with oxygen/ozone, UV light and elevated temperatures simultaneously, under conditions described in Example 2. A 1 ml portion of the stressed blood was reinjected to the same animal via the gluteal muscle. Such treatments took place on days 7, 8 and 20 following reception. Group B had blood withdrawn and reinjected in the same manner and in the same volumes, but the blood was not stressed (sham treatment). After 12 weeks from the last treatment, the animals were sacrificed by overdose of anaesthetic, and the arterial system was flushed with modified Krebs-Heinsleit (KH) solution. The iliac arteries were removed and preserved for in vitro vasoconstriction studies.

The arterial vessels were cleaned from all the fat and connective tissue, and rings (0.4 cm) were cut from the vessel. Rings, one endothelium denuded and one endothelium intact from each animal, were mounted onto wire stirrups, suspended in organ chambers (Radnoti Glass Technology) filled with oxygenated (95% 02/5% CO2) KH at 37 degrees C., and connected to force transducers (Harvard Apparatus) to record changes in isometric force. The output from the transducer was amplified, converted to digital signals and collected by Biopac data acquisition system MP100 (Harvard Apparatus). The rings were stretched to and maintained at a preload of 2 g and allowed to equilibrate for 2 hours. During the equilibration period, the buffer was changed every 30 minutes and continuously bubbled with 95% oxygen and 5% carbon dioxide. After equilibration, all aortic rings were exposed to cumulative concentartions of phenylephrine, a potent alpha-agonist (1×10⁻¹⁰ to 1×10⁻⁴ M) to determine contractile response. Then the rings were contracted with ED₆₅ of phenylephrine to obtain the maximal contraction, and then exposed to cumulative concentrations of acetylcholine (1×10⁻¹⁰ to 1×10⁴ M) to observe the relaxation result.

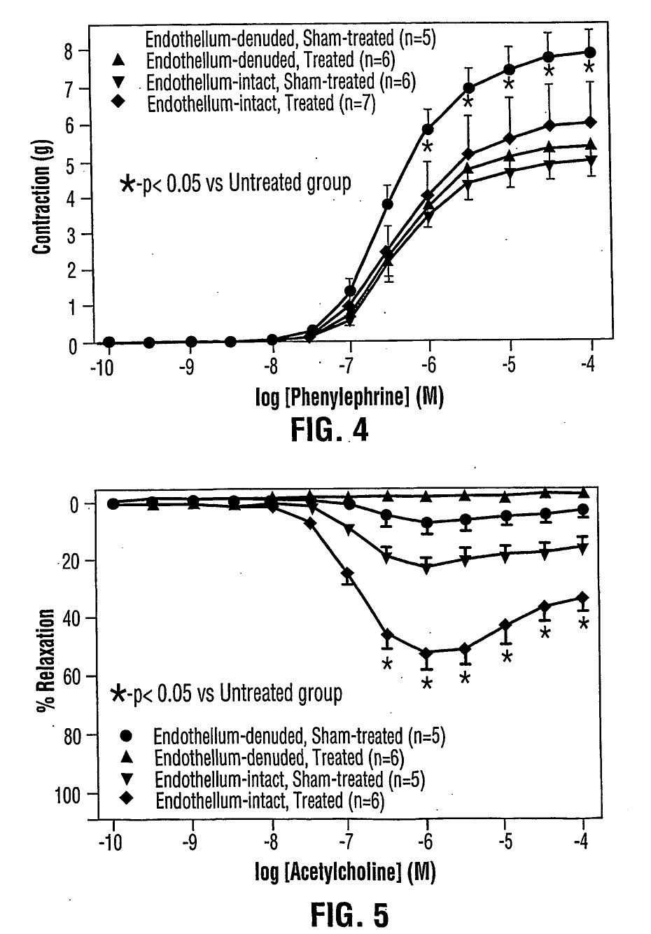

Vasoreactivity reaction to phenylephrine results are presented graphically on FIG. 4. The reaction of the endothelium-intact sample from the treated animals is significantly different from that of the sham treated animals. On the endothelium denuded samples, there is no significant difference between the treated animals and the sham treated animals.

FIG. 5 presents graphically the effet of relaxation induced by acetylcholine on the endothelium-intact and the endothelium-denuded iliac artery samples from treated and sham treated rabbits. The endothelium-denuded samples are significantly lower, showing the involvement of the endothelium in the process of the invention, effecting a significant improvement in endothelial function.

Values presented on FIGS. 4 and 5 are mean—s.e.

Since the stressed cell therapy as described herein has a beneficial effect on endothelial function, the therapy alone and in combination with other available treatments known to have similar beneficial effects on the endothelium, such as use of the pharmaceuticals as discussed herein, show potential in the treatment of neuro-degenerative disorders such as Alzheimer's Disease, Parkinson's Disease, and senile dementia.

What is claimed is:

1 Use in the preparation of a medicament for alleviation, prophylaxis against or preconditioning to hinder the on-set and progression of a neuro-degenerative disorder in a mammalian patient suffering from or at risk to contract such a disorder, of stressed autologous blood cells, said cells having been stressed by extracorporeal subjection to oxidative stress.

2. Use according to claim 1 wherein the cells have additionally been extracorporeally subjected simultaneously to UV light.

3. Use according to claim 1 or claim 2 wherein the oxidative stressor is exposure to a mixture of medical grade oxygen and ozone gas, with an ozone content up to about 300 μg/ml.

4. Use according to claim 3 wherein the oxygen/ozone gas mixture is bubbled through a suspension of blood cells at a rate of from 0.01-2.0 litres per minute (STP).

5. Use according to claim 4 wherein the suspension of blood cells is whole blood, of a volume from 0.1-100 ml.

6. Use according to any preceding claim wherein the blood cells are additionally subjected to elevated temperature of from 40-50° C., simultaneously with the subjection to oxidative stress.

7. Use according to any preceding claim in combination with adminisration to the patient of an effective amount of an ACE inhibitor.

8. Use according to claim 7 wherein the ACE inhibitor is selected from alacepril, benazepril, captopril, ceronapril, cilazapril, delapril, enalapril, enalaprilat, fosinopril, imidapril, lisinopril, moveltopril, perindopril, quinapril, ramipril, spirapril, temocapril and trandolapril.

9. Use according to any of claim 1-6 in combination with administration to the patient of an effective amount of an ACE inhibitor selected from alacepril, benazepril, captopril, ceronapril, cilazapril, delapril, enalapril, enalaprilat, fosinopril, imidapril, lisinopril, moveltopril, quinapril, ramipril, spirapril, temocapril and trandolapril.

10. Use according to any of claims 1-6 in combination with administration to the patient of an effective amount of an angiotensin II receptor antagonist.

11. Use according to claim 10 wherein the angiotensin II receptor antagonist is selected from candesartan, eprosartan, irbesartan, losartan and valsartan.

12. Use according to any of claims 1-6 in combination with administration to the patient of an effective amount of an effective amount of an inhibitor of the enzyme HMG CoA reductase.

13. Use according to claim 12 wherein the inhibitor of the enzyme HMG CoA reductase is atorvastatin, fluvastatin, lovastatin, simvastatin, pravastatin or cerivastatin.

14. Use according to any of claims 1-6 in combination with administration to the patient of an effective amount of an effective amount of a dihydropyridine-type calcium channel blocker drug.

15. Use according to claim 14 wherein the drug is amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine or nitrendipine.

16. A method of treating a patient to alleviate a neurological disorder suffered by the patient, which comprises altering the defective endothelium of the patient towards normalization of its function by administration to the patient of autologous blood cells which have been extracorporeally stressed by subjection to appropriate amounts of oxidative stress.

17. The method of claim 11 wherein the cells have also been stressed by simultaneous exposure to ultraviolet radiation and at an elevated temperature.

18. A method of treating a patient to alleviate a neurological disorder suffered by the patient, which comprises:
diagnosing patients to determine the presence in said patients of defective endothelial function in brain blood vessels of the patients;
selecting patients diagnosed with defective blood vessel endothelial function, and
administering to the selected patients autologous blood cells which have been extracorporeally stressed by subjection to appropriate amounts of oxidative stress.

19. The process of claim 18 wherein the autologous blood cells have additionally been stressed by simultaneous extracorporeal subjection to UV light.

20. Method according to claim 18 or claim 19 wherein the patient is additionally treated by administration of an ACE inhibitor, an angiotensin II receptor antagonist, an inhibitor of HMG CoA reductase, a dyhydropyridine calcium channel blocker or pentoxyfylline.
